(19) United States
(12) Plant Patent Application Publication
(10) Pub. No.: US 2020/0315078 P1
(43) Pub. Date: Oct. 1, 2020

(54) MANDARIN TREE NAMED 'PE1'

(50) ;

(71) Applicant: CITRIGENE (Pty) Ltd, Humansdorp (ZA)

(72) Inventor: Gert Ferreira, Patensie (ZA)

(73) Assignee: CITRIGENE (Pty) Ltd, Humansdorp (ZA)

(21) Appl. No.: 16/501,343

(22) Filed: Mar. 27, 2019

(51) Int. Cl. A01H 6/78

(52) U.S. Cl. PLT/202

(57) ABSTRACT

A new and distinct mandarin tree characterized by a ripening 2 3 weeks earlier than ‘Nadorcott’, having better rind quality (e.g., smoother rind, less prone to sunburn, less ridging, firmer peel, and a lighter color with an attractive blush), and a more round shape than ‘Nadorcott’.

Genus and species: Citrus reticulata.

Variety denomination: ‘PATENSIE EARLY’.

### BACKGROUND OF THE INVENTION

The present invention relates to a new and distinct variety of a mandarin tree named ‘Patensie Early’, which originated from a spontaneous mutant of ‘Nadorcott’ (U.S. Plant Pat. No. 10,480). The new mandarin variety has an earlier ripening time and improved rind quality as compared to ‘Nadorcott’.

### SUMMARY OF THE INVENTION

The following unique combination of characteristics that are outstanding in the new mandarin variety and distinguish it from ‘Nadorcott’, as well as from all other varieties of which I am aware:
1. Earlier ripening (2-3 weeks earlier than ‘Nadorcott’) due to earlier rind color development and lower acidity;
2. Better rind quality, namely smoother rind, less prone to sunburn, less ridging (radial grooves), firmer peel, lighter color with an attractive orange blush;
3. Less flattened and more round fruit shape compared with ‘Nadorcott’.

Additional details on how the two varieties can be distinguished is shown below.

| | ‘Nadorcott’ | ‘Patensie Early’ |
|---|---|---|
| Fruit Shape | Oblate to deeply oblate; | Slightly oblate; approximately |
| | typically 0.80 H:D | 0.85 H:D ratio of height to |
| | ratio with | diameter; rounder appearance |
| | flatter appearance | than ‘Nadorcott’ fruit |
| Fruit Height | ±48 mm | 37 to 53 mm |
| Fruit | ±60 mm | 42 to 62 mm |
| Diameter | | |
| Fruit Stem- | Radial grooves or ribbing | Ribbing is typically absent to |
| end | are present (FIG. 5) | very slight (see FIG. 5) |
| Skin | 2.8 to 3.5 mm | 2.5 to 3.0 mm |
| Thickness | RHS yellow-green 147B | RHS yellow-green 147B with |
| Rind color | | earlier color break than |
| immature | | ‘Nadorcott’ (see FIGS. 4 & 6) |
| Rind color | Deep orange (RHS orange | Deep orange (RHS orange N25B) |
| mature | N25B) with later color | (see FIG. 5); salmon pink albedo |
| | development than | (RHS orange 24D) |
| | ‘Patensie | |
| | Early’ (see FIGS. 4, 5, 6); | |
| | salmon pink albedo (RHS | |
| | orange 24D) | |
| Maturity | Late-June through early- | 2 to 3 weeks earlier than |
| period | August in South Africa | ‘Nadorcott’ due to earlier rind |
| | | color and lower acidity (June 6 |
| | | to July 25 in South Africa) |
| Eating | Very good at maturity, but | Typically +12° Brix and 1.0 to |
| quality (at | higher acidity when | 1.2% acidity and comparable |
| maturity) | ‘Patensie | with ‘Nadorcott’ |
| | Early’ is at | when ‘Nadorcott’ |
| | optimal maturity, | achieves maturity; lower acidity |
| | i.e., ‘Nadorcott’ is later | than ‘Nadorcott’ combined with |
| | maturing | earlier color development result |
| | | in earlier maturity than |
| | | ‘Nadorcott’ |

This new cultivar has been reproduced only by asexual propagation (by budwood), in Patensie, Eastern Cape South Africa. Each of the progeny exhibits identical characteristics to the original plant. Asexual propagation by budwood, as done in Patensie, Eastern Cape South Africa, shows that the foregoing characteristics and distinctions come true to form and are established and transmitted through succeeding propagations. The present invention has not been evaluated under all possible environmental conditions. The phenotype may change with variations in environment without a change in the genotype of the plant.

### BRIEF DESCRIPTION OF THE DRAWINGS

The accompanying illustration shows typical specimens of the leaves, flowers, and fruit of this new variety depicted in color as nearly true as it is reasonably possible to make the same in a color illustration of this character.

FIG. 1 shows a leaves on a ‘Patensie Eearly’ tree. The leaves of ‘Patensie Early’ are similar in leaf shape and size, but slightly paler in color, compared with leaves of ‘Nadorcott’ under the same condition.

FIG. 2 shows flowers on a of ‘Patensie Early’ tree, which are similar to those found on ‘Nadorcott’.

FIG. 3 shows fruit from ‘Patensie Early’ and ‘Nadorcott’ trees, demonstrating the earlier rind color development of ‘Patensie Early’ fruit compared with ‘Nadorcott’ fruit under the same growing conditions.

FIG. 4 shows fruit from ‘Patensie Early’, ‘Nova’ (unpatented) and ‘Tango’ (U.S. Plant Pat. No. 17,863) trees, demonstrating the early color break of the rind of ‘Patensie Early’ compared with ‘Tango’, but similar to ‘Nova’.

FIG. 5 shows fully mature fruit from ‘Patensie Early’ (upper) and ‘Nadorcott’ (lower) trees, demonstrating the earlier rind color development of ‘Patensie Early’ fruit compared with ‘Nadorcott’ fruit under the same growing conditions as well as less ribbing/radial grooves.

FIG. 6 shows ‘Patensie Early’ trees in a trial plot in Patensie, Eastern Cape Province, South Africa.

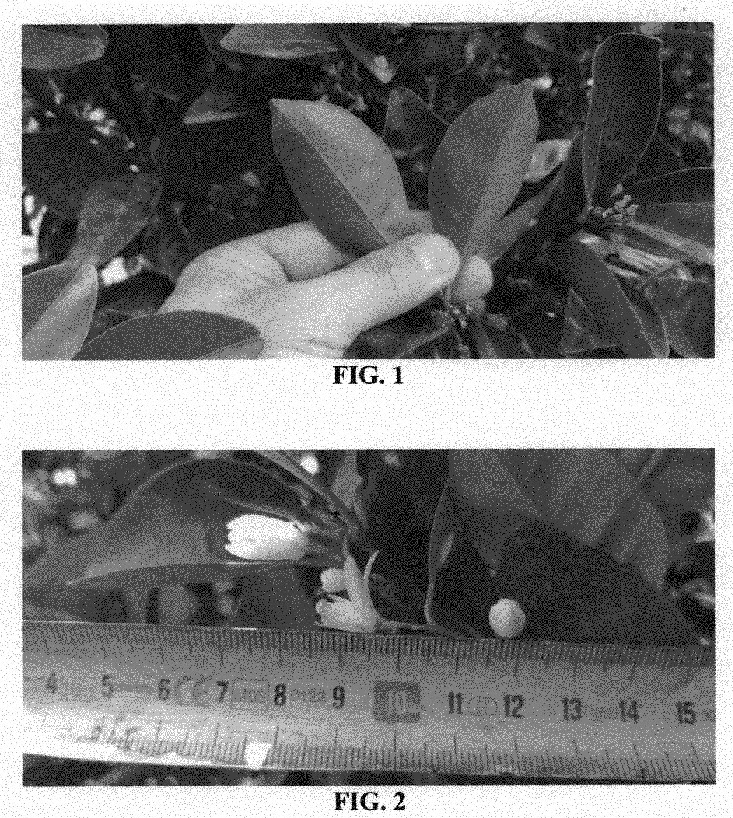

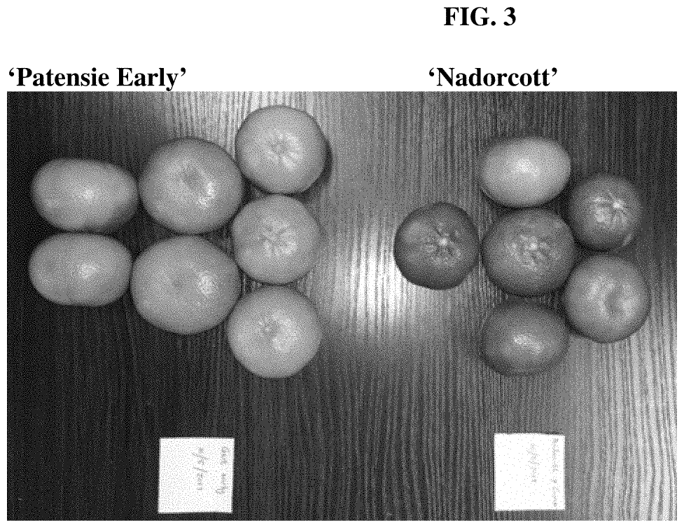

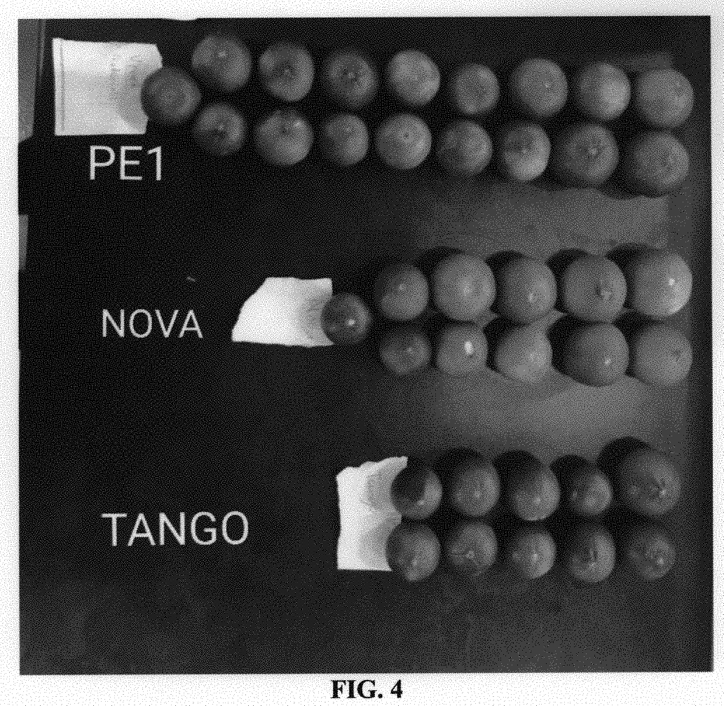

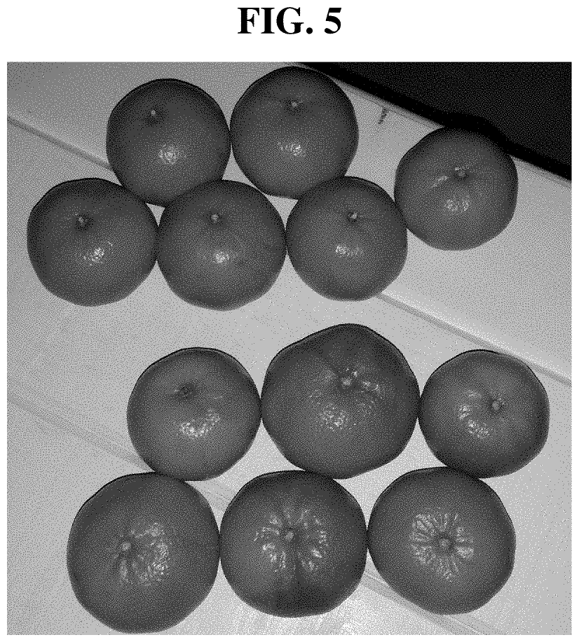

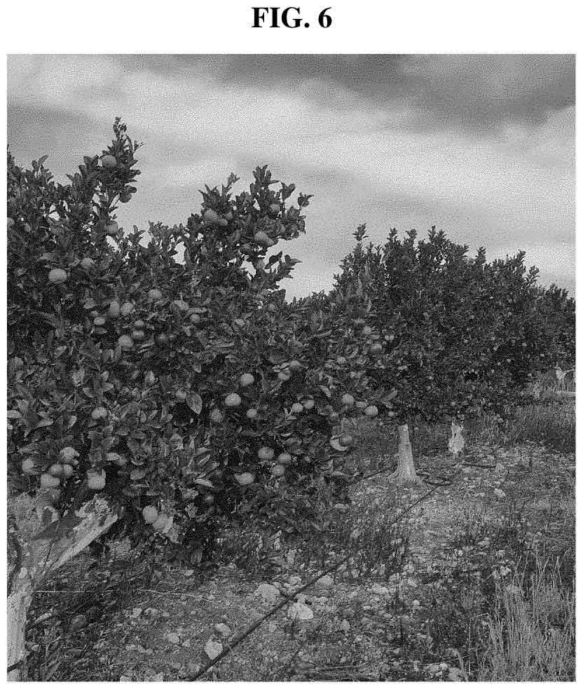

### DETAILED BOTANICAL DESCRIPTION

The following is a detailed description of the new mandarin based on observations taken of a 5 year-old specimen grown in trial beds outside in full sun in Patensie, Eastern Cape South Africa. The color descriptions are all based on The Royal Horticultural Society Colour Chart, 5^{th} edition, 2007.
Parentage: Spontaneous mutation of ‘Nadorcott’.
Tree:
   Ploidy.—Dipolid.
   Size.—Same as ‘Nadorcott’.
   Height.—3.5 m.
   Tree spread.—2.3 m.
   Vigor.—Strong.
   Density.—Open to medium, similar to ‘Nadorcott’ mandarin.
   Form.—Generally upright in the first 4 years followed by a tendency to grow into more spherical shape in the following years, similar to ‘Nadorcott’ mandarin.
Trunk:
   Trunk diameter.—17 cm in diameter at a height of 35 cm above the ground, 5 year old tree.
   Trunk texture.—Smooth, similar to ‘Nadorcott’ mandarin.
   Trunk bark color.—RHS grey-brown N199A, similar to ‘Nadorcott’ mandarin.
Branches:
   Branch length.—400 mm.
   Branch diameter.—170 mm.
   Branch texture.—Smooth.
   Branch color.—RHS grey-brown N199A.
   Strength.—Strong.
   Thorniness.—Typically absent, rarely short spines (3 to 6 mm in length).
Roots: The roots are on rootstock Swingle Citrumelo.
Leaves:
   Size (lamina average).—Length: 78 mm. Width: 34.3 mm.
   Shape.—Ellipical.
   Apex.—Acute to abtuse.
   Margin.—Crenate.
   Surface.—Upper surface: smooth. Lower surface: smooth.
   Color.—Upper surface (apaxial): RHS green 137A. Lower surface (abaxial): RHS yellow-green 146B; the leaves of ‘Patensie Early’ are similar in leaf shape and size, but slightly paler in color compared with leaves of ‘Nadorcott’ under the same condition.
   Petiole.—Shape: petiolate. Length: 9.2 mm. Diameter: 1.4 mm. Color: RHS Green 137C.
Flowers and flower buds:
   Type.—Clustered influorescent type.
   Bearing.—Once per year.
   Flower diameter.—6 mm.
   Flower height.—13 mm.
   Flower blooming period.—First bloom: Observed Aug. 15, Full bloom: Observed Sept 20 in Eastern Cape Province, Patensie
   Number of flowers.—Normal single and cluster, same as ‘Nadorcott’.
   Color.—RHS green-white 1570.
Rostellum:
   Length.—6 mm.
   Thickness.—1 mm.
   Color.—RHS Green 137A.
Fruit:
   Size.—Uniform.
   Shape.—Slightly oblate, approximately 0.85 ratio of height to width; less oblate and more round than ‘Nadorcott’ mandarin fruit.
   Tall.—37 to 53 mm.
   Width.—42 to 62 mm.
   Average weight (per individual fruit).—90 g.
   Texture.—Smooth, similar to ‘Nadorcott’ mandarin fruit.
   Stylar-end.—slightly depressed stylar end with no navel or stylar opening but an obvious areola.
   Color.—RHS orange N25B when fully mature, green fruit is RHS yellow-green 147B; early color development than ‘Nadorcott’.
   Harvesting.—First pick around October 6 (based on season and rootstock); last pick around July 25 (based on season and rootstock), in South Africa, Eastern Cape Province, Patensie. Approximately 3 weeks earlier than ‘Nadorcott’ under the same growing conditions.
   Productivity.—Good (30 to 50 kg per tree on 4- and 5-year-old trees), similar to ‘Nadorcott’.
Fruit skin:
   Adherence.—Easy peeling when fruit are mature.
   Thickness.—2.5 to 3.0 mm; typically slightly thinner than ‘Nadorcott’ mandarin.
   Texture.—Smooth with less ribbing/radial grooves at the stem-end compared with ‘Nadorcott’ fruit which typically have ribbing at the stem-end.
   Color.—Earlier rind color development compared with ‘Nadorcott’; Flavedo (epicarp) orange when fully mature (RHS orange N25B); Albedo (mesocarp): RHS orange 24D.
Fruit flesh:
   Flesh (pulp) texture.—Smooth.
   Flesh (pulp) color.—RHS orange-red N30D.
   Number of segments.—9 to 11.
   Juice content.—Typically +50% when fully mature.
   Eating quality.—Typically +12° Brix and 1.0 to 1.2% acidity at maturity with lower acidity than ‘Nadorcott’ when ‘Patensie Early’ is at optimal fruit maturity.
Seeds:
   Number.—Zero when not cross pollinate by compatible pollen; numerous seeds when cross-pollinated by compatible pollen.
   Embryony.—Polyembryonic.
   Seed coat color.—Grey-yellow 162D, similar to ‘Nadorcott’.

I claim:

1. A new and distinct variety of mandarin tree, substantially as herein shown and described.
